(19) United States
(12) Patent Application Publication
(10) Pub. No.: US 2021/0186038 A1
(43) Pub. Date: Jun. 24, 2021

(54) A METHOD OF STRAIN DEVELOPMENT

(71) Applicant: PAK GIDA ÜRETIM VE PAZARLAMA ANONIM SIRKETI, Istanbul (TR)

(72) Inventors: Sureyya Mert SELIMOGLU, Kartepe, Kocaeli (TR); Filiz ALEMDAR, Kartepe, Kocaeli (TR); Mustafa TURKER, Kartepe, Kocaeli (TR)

(21) Appl. No.: 17/054,225

(22) PCT Filed: Jan. 10, 2020
(86) PCT No.: PCT/TR2020/050016

§ 371 Date: Nov. 10, 2020

(30) Foreign Application Priority Data
Jan. 10, 2019 (TR) 2019/00376

(51) Int. Cl. A21D 8/04; A21D 2/08; C12N 1/18

(52) U.S. Cl. CPC A21D 8/04; C12N 1/18; A21D 2/08

(57) ABSTRACT

Disclosed is a method of development and selection of baker's yeast strains that can exhibit high raising activity in sweet and/or lean dough in the bakery and pastry industries. The method includes the steps of: (i) developing the baker's yeast strain, preferably with the process of multiple crossing, by using the combination of one or more than one of the mutagenesis, evolutionary engineering, asexual hybridization, recombinant DNA technology and sexual hybridization methods; (ii) preparing the synthetic or flour-based selective nutrient medium, preferably the synthetic nutrient medium having a sugar content of 0%, 15% and/or 25%, and incubating the strains developed in multiple well plates inside the nutrient medium prepared; (iii) scanning the strains incubated by using a high-resolution optical scanner, or by the naked eye or a device with a spectrophotometric reader, preferably by using a high-resolution optical scanner, and selecting the strains that perform the best acidification.

### TECHNICAL FIELD

The present invention relates to the process of selection of the baker's yeast from a high throughput platform, after said yeast is developed by using natural methods, wherein said yeast is capable of exhibiting high raising activity in sweet and/or lean dough in the bakery and pastry industries.

### PRIOR ART

The natural methods used for developing strains are collected under three main titles including; mutagenesis, sexual hybridization, and evolutionary engineering.

Mutagenesis: In this technique, the yeast is subjected to mutagenic agents such as EMS (ethyl methanesulfonate) (Demeke et al., 2013), MCB (methyl benzimidazole carbamate) (Zheng et al., 2013) or UV (ultraviolet light) (Salema-Oom et al.L, 2011). Therefore, it is ensured for random mutations to occur in their genome. The yeasts that have been subjected to mutagenic agents are subjected to a selection process in the media having the desired features in the next step. Thus, the selection of the yeast that shows the desired features is achieved.

Sexual Hybridization: Sexual hybridization is performed in three ways. These are called direct crossing, uncommon crossing and multiple crossing (genome mixing). The process of sporulation of the yeast and afterward crossing of different spores constitutes the essence of each of the three methods. In the direct crossing method; spores that have been selected under the microscope in a solid agar medium are isolated and hence the desired pairs are combined (Nakagawa and Ouchi, 1994). The uncommon crossing is rather based on; the principle of combining the strains that are not inclined to sporulation under pressure and hence directly merging them with the haploid having the desired character (Oda and Ouchi, 1990). In multiple crossing, the processes of a random combination of the strains sporulated and the selection of the strain having the desired character on a suitable platform are conducted (Higgins et al., 2001; Struyf et al., 2017).

Evolutionary Engineering: In this technique that can also be called directed evolution engineering, a cultivation process with a standard nutrient medium is started. Subsequently, the current nutrient medium is replaced quite slowly and gradually with a new nutrient medium (or a new component) to which the strain is desired to be adapted. Thus, it is ensured that the ones among the individuals in the generations developed from the strain fertilized which are capable of adapting to the new medium dominate the culture and that the new strains that can show the best adaptation to this new medium are produced (Teunissen et al., 2002).

In the bakery industry, two types of bread are essentially defined depending on the needs of the global market. In this respect, in addition to the basic inputs including the components of flour, water and salt, the breads that contain sugar at the ratio of 6-30%, and the margarine at the ratio of 2-12% are generally categorized as “sweet bread”. On the contrary, breads that contain sugar at the ratio of 0-6%, and the margarine at the ratio of 0-2% are categorized as “lean bread”. The yeast strains that have high raising activity in different types of doughs are developed by yeast producers due to said various needs and habits of consumers. In this regard, natural methods are entirely preferred within the frame of the current food regulations. The yeasts developed by using natural methods are subjected to high-throughput activity tests and in this way; the strain that has the highest activity is selected. During the implementation of these methods, flour-based dough formulations cannot be used because of the components they contain such as starch, etc. Therefore, “liquid synthetic dough” formulations are commonly preferred in the studies carried out (Myers et al. 1997; Bell et al. 2001; Higgins et al. 2001; Panadero et al. 2005). In the studies carried out within this context, the amount the yeast acidifies the synthetic dough that it contains inside is measured (Kara et al. 1988; Wick et al. 2001; Sigler 2013). By this means, interference for the yeast's sugar metabolism and the ability to produce CO₂ is indirectly obtained. The most important problem being faced during the use of liquid synthetic doughs is the potential of mutual affection of sugar metabolism and the CO₂ production on the acidification of the nutrient medium. This leads to doubts on the correct interpretation of the acidification profile obtained. Moreover, depending on the reproduction of the yeast cultivated in a growth culture for testing, an increase occurs in the turbidity of the liquid nutrient medium. In such cases, some problems can also occur depending on the method for determining of acidification. Furthermore, the liquid nutrient medium is not capable of completely simulating the rheological structure the yeast has. Therefore, there is a high risk that the possible dynamics that are likely to occur depending on the rheological structure during the selection process affects the yields to be obtained.

Although the sweet doughs that are preferred in the global market have a sugar content at the ratio of 6-30%, very high amounts of sugar concentrations (for example, 50%), are commonly preferred in current publications known from the literature, and this decreases the reliability of the selectivity of said synthetic dough. In the studies in which the reasonable rates in said range regarding the sugar content are preferred, the osmolarity values (or water activity) corresponding to the flour-based dough crossing cannot frequently be provided and this causes an additional problem in the correct assessment of yeast activity as well.

As well as the standard components, a preservative at the rate of 0.1-0.5% is added to the breads that are produced by industrial bread manufacturers. The most commonly used one among these preservatives is calcium propionate. Another drawback of the scanning methods known in the literature is that the calcium propionate is not added to the liquid synthetic dough formulations. It makes suspicious the compatibility of activities of the yeast strains that have been obtained during the scanning processes carried out with commercial dough formulations.

In conclusion, a novelty has been required to be developed in the related art due to the aforementioned drawbacks and the lack of current solutions for the subject matter.

### BRIEF DESCRIPTION OF THE INVENTION

The present invention relates to a method strain development that meets the aforementioned requirements, eliminates all of the disadvantages known from the prior art and provides some additional advantages to the related art.

The primary aim of the invention is to develop the baker's yeasts that can exhibit high raising activity in sweet and/or lean doughs used in the bakery and pastry industries by using natural methods, and afterward to provide a method of the selection of them on a high throughput platform by using synthetic dough formulations in an attempt to determine the yeast activity.

Another aim of the invention is to ensure the selection of high activity yeast strains through the use of the synthetic doughs in the method which are capable of simulating the rheological structure sweet and/or lean doughs have inside, by simulating the possible dynamics that are likely to occur depending on the rheological structure during the selection process.

The other aim of the invention is to provide the selection of the high activity yeast strains that perform the best acidification by ensuring the correct results are assessed in the determination of acidification due to the use of the synthetic doughs that minimize the effects of sugar metabolism and the CO₂ production on acidification of the nutrient medium.

An additional aim of the invention is to provide the development and selection of the yeast strains showing high activity in the presence of the preservatives that are used in sweet and/or lean doughs in the bakery and pastry industries because the synthetic doughs that are used in the method contain calcium-propionate inside.

To achieve the aforementioned aims, the invention is related to a method of development and selection of the baker's yeast strains that can exhibit high raising activity in sweet and/or lean doughs in the bakery and pastry industries, the method comprises the following steps of the process;
i. developing the baker's yeast strain, preferably with the process of multiple crossing, by using the combination of one or more than one of the mutagenesis, evolutionary engineering, asexual hybridization, recombinant DNA technology and sexual hybridization methods;
ii. preparing the synthetic or flour-based selective nutrient medium, preferably the synthetic nutrient medium with the sugar content of 0%, 15% and/or 25% and incubating the strains developed, in multiple well plates inside the nutrient medium prepared;
iii. scanning the strains incubated by using a high-resolution optical scanner, or by naked eye or a device with a spectrophotometric reader, preferably by using a high-resolution optical scanner, and selecting the strains that perform the best acidification which is to be determined based on the results of the analysis.

Structural and characteristic features in addition to all of the advantages of the invention will be understood more clearly through the detailed description given below and hence the evaluation should be done considering this detailed description.

### DETAILED DESCRIPTION OF THE INVENTION

In this detailed description, the method of strain development according to the invention has been disclosed only to provide a better understanding of the subject matter and this detailed description should not be construed to limit the scope of the invention.

The invention is related to the selection process of the baker's yeast that is capable of exhibiting high raising activity in sweet and/or lean dough in the bakery and pastry industries from a high throughput platform after said yeast is developed by using natural methods.

The principal aim of the process of subjecting the yeasts to high-throughput activity tests is to attempt to test sugar metabolisms of a plurality of yeasts at the same time and under the same conditions. Therefore, during these tests, the possible effects of other parameters on the results should be minimized. However, as the yeast cells are immersed in liquid in the scanning processes carried out at the site of the liquid growth culture, the CO₂ gas that has emerged as a result of sugar metabolism is directly released into the liquid. In such cases, the acidity of the growth culture is increased depending on the amount of CO₂ released and the instantaneous pH value of the growth culture, which is inevitable. In the nutrient medium subject to the invention, the yeast is cultivated in a semi-solid culture and most of the amount of CO₂ it has produced is released to the air. Thus, it can be possible to obtain more accurate results concerning sugar metabolism through minimum experimental disturbance.

With the development of biomass, the turbidity of the liquid nutrient medium is increased. It may be possible that the current turbidity during the process of reading (optical or spectrophotometric) that is to be performed at the end of the duration of cultivation has negative effects on analysis results.

Furthermore, the nutrient medium proposed within the scope of the invention is capable of much better simulating of the rheological structure of the dough in comparison to the liquid growth culture. Thus, it is ensured that the possible dynamics that are likely to occur depending on the rheological structure during the selection process can be simulated as well.

With the compositions of the semi-solid nutrient medium that are disclosed in detail within the scope of the invention, the dough prescriptions that have commonly been preferred in the global market can be simulated by ensuring optimum conditions.

Three different types of nutrient media are defined in the method according to the invention:
1. Synthetic Lean Dough (SLD): Synthetic lean dough nutrient medium.
2. 15% Sugared Synthetic Dough (15S-SD): 15% sugared synthetic dough nutrient medium.
3. 25% Sugared Synthetic Dough (25S-SD): 25% sugared synthetic dough nutrient medium.

Contrary to other similar dough, with the use of these nutrient media, the same osmolarity (or water activity) values with those of the doughs that contain the equivalent amounts of sugar can be obtained (Table 1). Thus, it is enabled to perform the selection process on a much more reasonable platform in comparing the yeast activities of different types of strains.

TABLE 1
Content and water activity values of flour-based doughs
and synthetic derivatives thereof
Flour-Based Dough

| | With 0% | With 15% | With 25% |
|---|---|---|---|
| | Sugar | Sugar | Sugar |
| | Content | Content | Content |
| Components | (a_{w}: 0.97) | (a_{w}: 0.94) | (a_{w}: 0.91) |
| Wheat flour | %60.52 (w/w) | %58.74 (w/w) | %56.53 (w/w) |
| Sugar | — | %8.81 (w/w) | %14.13 (w/w) |
| Margarine | — | %3.92 (w/w) | %5.65 (w/w) |
| Salt | %0.91 (w/w) | %0.88 (w/w) | %0.85 (w/w) |
| Calcium Propionate | %0.24 (w/w) | %0.23 (w/w) | %0.23 (w/w) |
| Water | %38.33 (v/w) | %27.41 (v/w) | %22.61 (w/w) |

Synthetic Flour

| | S-LD | 15S-SD | 25S-SD |
|---|---|---|---|
| Components | (a_{w}: 0.97) | (a_{w}: 0.94) | (a_{w}: 0.91) |
| (1) Concentrated | — | %20 (v/v) | %20 (v/v) |
| Nutrient Solution | | | |
| (2) Maltose Indicator | %7.00 (w/v) | — | — |
| Nutrient Medium | | | |
| (3) Yeast Extract | — | %0.50 (w/v) | %0.50 (w/v) |
| (4) Glucose | — | %3.00 (w/v) | %3.00 (w/v) |
| (5) Maltose | — | %9.00 (w/v) | %9.00 (w/v) |
| (6) Sorbitol | %13.00 (w/v) | %10.00 (w/v) | %16.00 (w/v) |
| (7) Sucrose | — | %15.00 (w/v) | %25.00 (w/v) |
| (8) Agar | — | %2.00 (w/v) | %2.00 (w/v) |
| (9) Calcium- | %0.24 (w/v) | %0.24 (w/v) | %0.24 (w/v) |
| propionate | | | |
| (10) pH Indicator | %0.90 (v/v) | %0.90 (v/v) | %0.90 (v/v) |
| (11) Water | %78.86 (v/v) | %39.36 (v/v) | %23.36 (v/v) |

a_{w}: Water activity values of flour-based dough and synthetic counterparts thereof after gelling. The values obtained show the average of measurements by three separate repetitions of the samples prepared at three separate times.
(w/w): expresses the ratio of weight/weight.
(v/w): expresses the ratio of volume/weight.
(v/v): expresses the ratio of volume/volume.
(w/v): expresses the ratio of weight/volume.

In addition to these, the commonly preferred calcium propionate has also been included by industrial manufacturers in the semi-solid nutrient medium formulations that are disclosed within the scope of the invention (Table 1). Thus, it is anticipated that the strains exhibiting high activity in the scanning processes to be carried out by using these nutrient media are also resistant to the additives used by industrial bread manufacturers and exhibit the same activity in the presence of these additives as well. This allows the selection processes to be performed using said nutrient media to be suitable for much broader-based applications.

The characteristics of the components used in synthetic dough (Table 1: SLD, 15S-SD, 25S-SD) produced to simulate three different nutrient media that are mentioned in the method according to the invention are disclosed below.

Concentrated Nutrient Solution (1), (Panadero et al. 2005), It is a mixture that contains the required vitamins and minerals for yeast reproduction. It is prepared in a 5× Concentrated form. Accordingly, it is prepared by dissolving the components of weight/volume 2% (w/v) MgSO₄.7H₂O (magnesium sulfate heptahydrate); 0.8% (w/v) KCl (potassium chloride); 4.7% (w/v) (NH₄)2HPO₄ (ammonium phosphate dibasic); 0.0016% (w/v) Thiamine; 0.0016% (w/v) Pyridoxine; 0.016% (w/v) Nicotinic acid in a 0.75M Citrate Buffer. The final pH of the solution is set to 5.5 with the addition of NaOH (sodium hydroxide). It is used after being sterilized with a 0.2 μm sterilized filter. It is used for meeting the basic food and mineral needs of yeast cells.

Maltose Indicator Nutrient Medium (2), (Baensch et al. 2009), It is used for evaluating the rates of maltose sugar intake of yeast cells. It is prepared by agitating the components of weight/volume 1% (w/v) Yeast extract, 2% (w/v) Peptone; 2% (w/v) Maltose and 2% Agar (w/v) in distilled water.

Yeast Extract (3), It is the cell extract that has been obtained after separating the cell wall of the yeast. It is used for feeding bacteria and yeast cells in addition to its usage as additives and sweeteners. In the studies carried out within the scope of the invention, it is used for meeting the basic nutrition needs of yeast cells.

Glucose (4), It is a six-carbonate monomeric structured sugar having the molecular formula of C₆H₁₂O₆. It is used by living creatures to meet their basic energy needs. In the context of the invention, it is used for meeting the basic nutrition needs of yeast cells.

Maltose (5), It is a disaccharide composed of two glucose monomers bound to each other with the link of α(1→4). It is also called malt sugar or maltobiosis. Yeasts firstly take the maltose into the cell through the maltose permease enzyme they have and then metabolize it with the maltase enzyme. The basic patterns of the starch obtained from the flour are maltose structured. Therefore, the yeast added to the dough can meet most of the amount of energy it requires through the starch obtained from the flour. The maltose used in the scope of the invention is used for simulating the starch obtained from the flour and for selecting the maltose metabolizing rate of the yeast.

Sorbitol (6), It is a type of sugar alcohol having a sweet flavor. It is quite slowly digested by human metabolism. The yeasts cannot digest sorbitol and it has not been observed that it has any toxic effects on the yeasts. Due to its characteristics, it is used as an osmolarity promoter in synthetic nutrient medium formulations. Such a characteristic of sorbitol has been used in the context of the invention.

Sucrose (7), It is known as table sugar as well. It is used both by industrial bread manufacturers and also by individual consumers in making of the breads that contain different rates of sugar. It is composed of two monomers including glucose and fructose. The yeasts receive sucrose into cells after separating the sucrose into their monomers through the invertase enzyme they release. It is used for simulating the breads that contain different concentrations of sugar within the scope of the invention.

Agar (8), It is composed of a linear-structured agarose polymer and a heterogeneous mixture of agaropectin molecules having a smaller structure. As it is not possible to be metabolized (because of its inert nature) by many microorganisms, it is used as a gelling agent in nutrient media. In the context of the invention, it has been attempted to simulate the viscoelastic structure of the dough in the selective nutrient medium by using this characteristic of agar. Moreover, it has been enabled to attain measurable inferences during the scanning tests carried out as to the yeast metabolism with the use of a suitable pH indicator.

Calcium-propionate (9), It is a type of additive listed with the code of E282. It is added as an additive to doughs by industrial bread manufacturers in making bakery products such as breads, etc. It is used for preventing mold formation; however, it is also known that it inhibits yeast reproduction to an extent. Within the framework of the invention, it is used for the selection of the suitable yeast strains for commercial bread dough formulations.

pH Indicator (10), It is a halochromic chemical component. It enables changes occurring in the pH (acidity-alkalinity rates) of the solution to which it is added to be determined through visual or spectrophotometric measurement methods. The intensity of the light it absorbs and/or reflects depending on the changes occurring in the pH of the medium is changed; thus, the pH values that are obtained through the naked eye, an optical reader or a spectrophotometer can be measured. In the scope of the invention, it is used for measuring the acidification occurring in the gel to be used for scanning. water (11), It is necessary for the yeast to remain alive and to reproduce, and is used for providing the nutrient medium having the suitable water activity values at which the yeast is viable.

The method according to the invention is essentially formed of three main steps of the process:
A. Strain Development
B. Incubation in Selective Nutrient Medium
C. Analysis and Selection

A. Strain Development

After a crossing process is carried out in the scope of multi crossing, the colonies that exhibit rapid growth in the YPD (yeast peptone dextrose) agar nutrient medium are selected. In this way, the preselection process of the strains that are most probably polyploid (having two or more chromosome copies) has been performed. At this point, it is passed to the scanning process in a selective nutrient medium for scanning metabolic activities of the strains obtained.

In addition to the process of multi crossing which is one of the sexual hybridization methods, other sexual hybridization methods, mutagenesis, evolutionary engineering, asexual hybridization (protoplast fusion), recombinant DNA technology methods or the combination of one or more than one of these methods can be used in the scope of the invention.

The asexual hybridization (protoplast fusion) mentioned herein means merging of the protoplast of any two cells with the genetic material contained in its content by use of a method such as chemical fusion, electrofusion, etc. (Oya et al. 1988, 1990; Jacobson and Trivedi 1990; Endo et al. 1995; Endo 1997; Takahashi and Motoki 2005; Takahashi et al. 2006).

The DNA technology mentioned herein means transforming a part of the genetic material of the cell through the use of a variety of synthetic biology methods and/or adding genetic materials to the cell externally (Rogers and Szostak 1987; Osinga et al. 1989, 1993; Van Rooijen et al. 1999; Bartolucci et al. 2013; Xiao et al. 2013; Zhang et al. 2013, 2014a,b,c,d).

B. Incubation in Selective Nutrient Medium

### a) Preparation of Selective Nutrient Medium

Synthetic Lean Dough (SLD):

In an embodiment of the invention, synthetic lean dough; has been formulated to simulate the physicochemical structure of lean doughs. In this context, 7 gr maltose indicator nutrient medium numbered 2, 13 gr sorbitol numbered 6 and 243 mg calcium propionate numbered 9 are mixed. The mixture obtained is autoclaved for 15 minutes at 121° C. after being dissolved in approximately 80 ml distilled water. 900 μl from the pH indicator numbered 10 is added to the nutrient medium autoclaved in a sterilized way. The rest of the solution is completed with the addition of 100 ml distilled water and afterward, the nutrient medium is cast in the plate. In the invention, contrary to the studies currently known from the literature, casting is performed in multi-well plates. The multi-well plates mentioned in the invention refer to the use of the plates having more than one well (e.g. 4, 6, 8, 12, 16, 24, 48, 96, 384 and 1536 wells). In this context, the use of 48-well plates has been preferred. During casting the selective nutrient medium in multi-well plates, it should be ensured that the volume cast in every well is equal. After casting the synthetic nutrient media in the multi-well plates, the plates are left to rest for approximately 30-60 mins to be ready for gelling and use. The plates obtained after the polymerization of gels are ready for use.

The S-SD nutrient medium with its formulation given in the framework of the invention can be prepared as disclosed below. Accordingly:

A basal nutrient medium is obtained by mixing 500 mg yeast extract numbered 3, 3 g glucose numbered 4, 9 g maltose numbered 5, 10 g sorbitol numbered 6, 2 g agar numbered 8 and 236 mg calcium propionate numbered 9. The mixture obtained is autoclaved for 15 mins at 121° C. after being dissolved in approximately 30-40 ml distilled water. 900 μl pH indicator numbered 10 and 20 ml 5× concentrated nutrient medium numbered 1 is added to the basal nutrient medium autoclaved in a sterilized way. The rest of the solution is completed with the addition of 100 ml distilled water and afterward, the nutrient medium is cast in the plate. In the invention, contrary to the studies currently known from the literature, casting is performed in multi-well plates. In this context, the use of 48-well plates has been preferred. The plates obtained after the polymerization of gels are ready for use.

15% Sugared Synthetic Dough (15S-SD):

In an embodiment of the invention, 15% sugared synthetic dough; has been formulated to simulate the physicochemical structure of doughs with 15% sugar content. In this context, a “15S-SD basal nutrient medium” is obtained by mixing 500 mg yeast extract numbered 3, 3 g glucose numbered 4, 9 g maltose numbered 5, 10 g sorbitol numbered 6, 15 g sucrose numbered 7, 2 g agar numbered 8 and 236 mg calcium propionate numbered 9. The mixture obtained is autoclaved for 15 minutes at 121° C. after being dissolved in approximately 30-40 ml distilled water. 900 μl from the pH indicator numbered 10 and 20 ml 5× concentrated nutrient medium numbered 1 is added to the 15S-SD basal nutrient medium autoclaved in a sterilized way. The residual volume of the solution is completed with the addition of 100 ml distilled water and afterward, the 15S-SD nutrient medium is cast in the plate. In the invention, contrary to the studies currently known from the literature, casting is performed in multi-well plates. In this context, the use of 48-well plates has been preferred. During casting the selective nutrient medium in multi-well plates, it should be ensured that the volume cast in every well is equal. After casting the synthetic nutrient media in the multi-well plates, the plates are left to rest for approximately 30-60 mins to be ready for gelling and use. The plates obtained after the polymerization of gels are ready for use.

25% Sugared Synthetic Dough (25S-SH):

In an embodiment of the invention, 25% sugared synthetic dough; has been formulated to simulate the physicochemical structure of doughs with 25% sugar content. In this context, a “25S-SD basal nutrient medium” is obtained by mixing 500 mg yeast extract numbered 3, 3 g glucose numbered 4, 9 g maltose numbered 5, 16 g sorbitol numbered 6, 25 g sucrose numbered 7, 2 g agar numbered 8 and 236 mg calcium propionate numbered 9. The mixture obtained is autoclaved for 15 minutes at 121° C. after being dissolved in approximately 30-40 ml distilled water. 900 μl from the pH indicator numbered 10 and 20 ml 5× concentrated nutrient medium numbered 1 is added to the 25S-SD basal nutrient medium autoclaved in a sterilized way. The residual volume of the solution is completed with the addition of 100 ml distilled water and afterward, the 25S-SD nutrient medium is cast in the plate. In the invention, contrary to the studies currently known from the literature, casting is performed in multi-well plates. In this context, the use of 48-well plates has been preferred. During casting the selective nutrient medium in multi-well plates, it should be ensured that the volume cast in every well is equal. After casting the synthetic nutrient media in the multi-well plates, the plates are left to rest for approximately 30-60 mins to be ready for gelling and use. The plates obtained after the polymerization of gels are ready for use.

The nutrient media SLD, 15S-SD and 25S-SD with their formulations given within the scope of the invention can be prepared without adding the component agar numbered 8 as well. The nutrient media prepared in this way are used in liquid form.

As well as the nutrient media SLD, 15S-SD and 25S-SD with their formulations given within the scope of the invention, the flour-based nutrient media with 0%, 15% and 25% sugar content can also be used for the same purpose.

48-well plates have been used in scanning processes carried out within the framework of the invention. As an alternative to this, 1, 4, 6, 8, 12, 16, 24, 48, 96, 384 and 1536-well plates can also be used.

### b) Incubation

At this step, the strains that have been preselected at the first step are cultivated in the selective nutrient medium for determining their activities by use of an indirect method and hence for making a comparison between the strains. The cultivated strains are kept in incubation 2-3 days at +30° C. for.

In the context of the invention, while it is possible to determine activities of the preselected strains by using indirect methods, they can be determined through direct methods as well. To this end, while it is possible to keep the cultivated strains in incubation for 2-3 days at +30° C., they can be left to rest for a period in the range of 10 minutes to 4 days at the temperature of 0° C. and +35° C.

C. Analysis and Selection

After incubation, to compare the pH changes occurring in the nutrient media contained in the wells, a scanning process is performed in the multi-well plates by using a high-resolution optical scanning system. Scanning results are processed through the use of an image processing computer program and hence the strains that have performed the best acidification are determined.

After incubation, by analyzing the pH changes in the nutrient media contained in the wells, a determination for the yeast metabolism can be performed indirectly. Moreover, it is also possible to use the nutrient media with their formulations, given within the framework of the invention, with MicroResp—a rapid microtiter plate system for carbon dioxide measurement (Campbell et al., 2003). This system has been subjected to the patent documents of Davidson et al. (2003, 2005). In this way, CO₂ release values of each strain can be directly attained. The use of said system with said dough formulations is also included in the scope of the invention.

A high-resolution optical scanner can be used for the scanning process of the invention, furthermore, an assessment with the naked eye or a reading process can also be performed through the use of a device having a spectrophotometric reader.

The method subject to the invention with its details mentioned above bears a similarity with the crossing method disclosed in Sante (2017), Gendelman et al. (2018) regarding the process of obtaining new yeast strains included in its scope. Furthermore, the method of the invention uses a synthetic dough formulation to detect the yeast activity, which constitutes another similarity between said two methods. However, the use of multi-well plates for selection, the composition of the selective nutrient media (SLD, 15S-SD and 25S-SD synthetic doughs) used with the multi-well plates, other tools and materials used for selection, and the methods of analysis and selection create a difference between said methods.

It has a similarity as it also comprises the sexual crossing methods disclosed in Endo et al. (1995), Endo (1997); the conventional crossing methods disclosed in Jean-Marc et al. (2015), however, it bears a difference regarding the other steps of the process.

It has a similarity as it also comprises the strain development with mutagenesis disclosed in Watanabe et al. (1992), Tamura et al. (1995), Ando et al. (1996), Endo (1997), Takada et al. (1998), Domingues et al. (1999), Sakurai et al. (2004) however, it bears a difference regarding the other steps of the process.

It bears a similarity with the step of strain development regarding the strain development with mutagenesis disclosed in Kyogoku et al. (1993) while it shows similarities with the composition of the 15S-SD and 25S-SD nutrient media regarding the use of yeast extract numbered 3, sucrose numbered 7, agar numbered 8 and Bromocresol purple as pH indicator numbered 10 in the nutrient medium composition used for selection. However, it has a difference regarding the use of multi-well plates for selection, other compositions of the selective nutrient media (SLD, 15S-SD and 25S-SD synthetic doughs) used with the multi-well plates, other tools and materials used for selection, and the methods of analysis and selection.

It has a similarity in terms of the strain development with a combined application of the multiple crossing method of the mutagenesis and sexual hybridization disclosed in Hamada et al. (1995), however, it bears a difference regarding the other steps of the process.

It has a similarity in terms of the strain development with a combined application of the direct crossing method of the mutagenesis and sexual hybridization disclosed in Sano et al. (2000), however, it bears a difference regarding the other steps of the process.

It has a similarity in terms of the strain development with the mutagenesis and other crossing methods disclosed in Bartolucci et al. (2016), and the use of the multi-well plates for selection. However, it bears differences regarding the composition of the selective nutrient media used with the multi-well plates, other tools and materials used for selection, and the methods of analysis and selection.

It has a similarity in terms of the strain development with a combined application of the methods of the mutagenesis and recombinant DNA technology disclosed in Maruyama et al. (1997), Takada et al. (1998), however, it bears a difference regarding the other steps of the process.

It has similarities with the step of strain development regarding a combined application of the direct crossing method s of mutagenesis and sexual hybridization disclosed in Gysler et al. (1995), the use of a synthetic dough formulation for detecting fermentative capacities of the yeast strains, and with the selective nutrient media (SLD, 15S-SD and 25S-SD) regarding maltose numbered 5 contained in the composition of the selective nutrient medium. However, it has a difference regarding the use of multiple well plates for selection, other compositions of the selective nutrient media (SLD, 15S-SD and 25S-SD synthetic doughs) used with the multiple well plates, other tools and materials used for selection, and the methods of analysis and selection.

It has a similarity in terms of the strain development with the direct crossing method of the sexual hybridization disclosed in Oda and Ouchi (1991), Takano et al. (1994, 2000), Domingues (1996), Tamura and Moriya (1997), Endo (1997), Ando et al. (2000), Gysler and Niederberger (2002), however, it bears a difference regarding the other steps of the process.

It has similarities with the step of strain development regarding the strain development with the direct crossing method of the sexual hybridization disclosed in Takano et al. (1990), the use of a synthetic dough formulation for detecting the yeast activity, and with the selective nutrient media (SLD, 15S-SD and 25S-SD) regarding maltose numbered 5 contained in the composition of the selective nutrient medium. However, it has a difference regarding the use of multi-well plates for selection, other compositions of the selective nutrient media (SLD, 15S-SD and 25S-SD) used with the multi-well plates, other tools and materials used for selection, and the methods of analysis and selection.

It has a similarity in terms of the strain development with the methods of recombinant DNA technology disclosed in Rogers and Szostak (1987), Osinga et al. (1989, 1993), Van Rooijen et al. (1999), Bartolucci et al. (2013), Xiao et al. (2013), Zhang et al. (2013, 2014a,b,c,d); the strain development with the combined application of the methods of recombinant DNA technology disclosed in Domingues (1994a,b) and the direct crossing method of sexual hybridization, however, it bears a difference regarding the other steps of the process.

It bears similarities with the SLD nutrient medium regarding the selective nutrient medium composition disclosed in Baensch et al. (2009), the maltose indicator nutrient medium numbered 2 and Bromcresol red as the pH indicator numbered 10; with the 15S-SD and 25S-SD nutrient media regarding yeast extract numbered 3, maltose numbered 5, agar numbered 8 and Bromcresol red as the pH indicator numbered 10. However, it has a difference regarding the use of multi-well plates for selection, other compositions of the selective nutrient media (SLD, 15S-SD and 25S-SD) used with the multi-well plates, other tools and materials used for selection, and the methods of analysis and selection.

It has a similarity regarding the strain development with the method of asexual hybridization (protoplast fusion) disclosed in Oya et al. (1988, 1990), Jacobson and Trivedi (1990), Endo et al. (1995), Endo (1997), Takahashi and Motoki (2005), Takahashi et al. (2006), however, it bears a difference regarding the other steps of the process.

It has a similarity regarding the strain development with the uncommon crossing method of sexual hybridization disclosed in Yamauchi et al. (1995), however, it bears a difference regarding the other steps of the process.

It has a similarity regarding the strain development with the method of multiple crossing of sexual hybridization disclosed in Bell and Bissinger (1996), Takada et al. (2005), however, it bears a difference regarding the other steps of the process.

### PATENTS AND RESOURCES REFERRED

ANDO MASAYASU; NAKAJIMA RYOICHI; HAMADA KAZUHIRO. (1996). New yeast and production of breads by utilizing the same yeast. JPH08332084 (A).

ANDO MASAYASU; SHIMIZU NATSUKO; SHINOMIYA YOSHIAKI. (2000). Ultra sugar resistant yeast for preparation of confectinary and bread. JP2000262275 (A).

BAENSCH JOHANNES; GYSLER CHRISTOF; NIEDERBERGER PETER. (2009). Low-temperature inactive industrial baker's yeast and process for preparing same. CN101343617 (A).

BAENSCH, J, GYSLER, C, NIEDERBERGER, P. (2009). Low-temperature inactive industrial baker's yeast and process for preparing same. CN patent: Cn101343617 (A).

BARTOLUCCI JEAN-CHARLES; COLAVIZZA DIDIER; LEGROS MELANIE; PIGNEDE GEORGES. (2013). Novel bread yeast strains. US2013344197 (A1).

BARTOLUCCI, JEAN-CHARLES; FONCHY-PENOT EVELYNE; LAGOUTTE YALCIN ILKNUR; PARASIE GEORGES; PETROFF DOMINIQUE; QUIPOURT-ISNARD ANNE-DOMINIQUE; TRIONE VALERIE (2016). Breadmaking yeast strains which are effective on non-sweetened or slightly sweetened dough. U.S. Pat. No. 10,015,972 (B2).

BELL PHILLIP JOHN LIVINGSTON; BISSINGER PETER HANS. (1996). High sugar dough yeast strains. AU5805696 (A).

BELL, P J L, HIGGINS, V J V E ATTFIELD, P V. (2001). Comparison of fermentative capacities of industrial baking and wild-type yeasts of the species saccharomyces cerevisiae in different sugar media. Letters in applied microbiology 32: 224-29.

CAMPBELL, C D, CHAPMAN, S J, CAMERON, C M, DAVIDSON, M S V E JACQUELINE M P. (2003). A rapid microtiter plate method to measure carbon dioxide evolved from carbon substrate amendments so as to determine the physiological profiles of soil microbial communities by using whole soil. Applied and environmental microbiology 69(6): 3593-99.

DAVIDSON, M S, CAMPBELL, C D V E CHAPMAN S J. (2003). Apparatus And Method. US2003024330 A1.

DAVIDSON, M S, CAMPBELL, C D V E CHAPMAN S J. (2005). A connection device for use with multi-well plates. UK patent: GB2410797 A.

DEMEKE M M, DIETZ H, LI Y VE DI\check{G}ERLERI. (2013). “Development of a d-xylose fermenting and inhibitor tolerant industrial saccharomyces cerevisiae strain with high performance in lignocellulose hydrolysates using metabolic and evolutionary engineering”. Biotechnol Biofuels 6: 89.

DOMINGUES, DAVID J; ATWELL, WILLIAM A; PILACINSKI, WILLIAM P. (1999). Yeast-Leavened Refrigerated Dough Products. U.S. Pat. No. 5,939,109 (A).

DOMINGUES, DAVID J. (1994B). Yeast-Leavened Refrigerated Dough Products. CA2616949 (A1).

DOMINGUES, DAVID J. (1996). Catabolite Non-Repressed Substrate-Limited Yeast Strains And Method Of Making. U.S. Pat. No. 5,508,047 (A).

DOMINGUES, DAVID, J. (1994A). Yeast-Leavened Refrigerated Dough Products. WO9419955 (A1).

ENDO HISANORI; HIYOSHI SADAYUKI; HIROSE DAIHACHI; SUGIYAMA YOKO. (1995). Novel Baker's Yeast. JPH07203952 (A).

ENDO, HISANORI. (1997). Freeze-Resistant Baker's Yeast Having Sugar Resistance. CA2153559 (C).

GENDELMAN, M., COHEN, T., MOR, S., KHUTORIAN, M. (2018). Freeze-Resistant Yeast And Uses Thereof. EP3318646 (A1).

GYSLER CHRISTOF; NIEDERBERGER PETER. (2002). Method For Introducing Recessive Properties Into The Genetic Background Of industrial Baker's Yeast. US2002004243 (A1).

GYSLER, CHRISTOF; HOTTINGER, HERBERT; NIEDERBERGER, PETER. (1995). Polyploid baker's yeasts having a low temperature inactivation property. U.S. Pat. No. 5,399,492 (A).

HAMADA KAZUHIRO; ANDO MASAYASU; YAMATO YUKA; SHIMIZU NATSUKO. (1995). Bread Yeast. JPH07123975 (A).

HIGGINS V J, BELL P J, DAWES I W, ATTFIELD P V. (2001). “Generation of a novel saccharomyces cerevisiae strain that exhibits strong maltose utilization and hyperosmotic resistance using nonrecombinant techniques”. Appl Environ Microbiol 67:4346-8.

JACOBSON, GUNNARD K; TRIVEDI, NAYANKUMAR B. (1990). Yeast strains, method of production and use in baking. U.S. Pat. No. 4,973,560 (A).

JEAN-MARC, LADRIERE; BARTOLUCCI, JEAN-CHARLES; FABIENNE, SUCHER; BENOIT, THOMAS. (2015). Saccharomyces cerevisiae strain suitable for bakery yeast manufacture, osmotolerant and manifesting heritable resistance to weak organic acids (versions) and such strain usage. U.S. Pat. No. 9,138,006 (B2).

KARA, B V, SIMPSON W J VE HAMMOND, J R M. (1988). Prediction Of The Fermentation Performance Of Brewing Yeast With The Acidification Power Test. J. Inst. Brew. (94): 153-58.

KYOGOKU YASUHISA; KAWASAKI HIDENORI; OUCHI KOZO. (1993). Baking Process. JPH05336872 (A).

MARUYAMA HIROYUKI; KYOGOKU KENJI; MATSUMOTO KEIJI; ASAHI KOJI; OYA KOZO; HARASHIMA TAKASHI; OSHIMA TAIJI. (1997). New Gene And New Yeast Containing The Gene. JPH09272 (A).

MYERS, D K, LAWLOR, D T M V E ATTFIELD, P V. (1997). Influence of invertase activity and glycerol synthesis and retention on fermentation of media with a high sugar concentration by saccharomyces cerevisiae. Applied And Environmental Microbiology 63(1): 145-50.

NAKAGAWA S, OUCHI K. (1994). “Construction from a single parent of baker's yeast strains with high freeze tolerance and fermentative activity in both lean and sweet doughs”. Appl Environ Microbiol 60: 3499-502.

ODA Y, OUCHI K. 1990. Hybridization of bakers' yeast by the rare-mating method to improve leavening ability in dough. Enzyme Microbe Technol 12: 989-93.

ODA YUJI; OUCHI KOZO. (1991). New baker's yeast and production of bread using same yeast. JPH0380073 (A).

OSINGA KLAAS ANNE; BEUDEKER ROBERT FRANCISCUS; VAN DER PLAAT JOHANNES BERTUS; DE HOLLANDER JOHANNES ABRAHAM. (1989). New Yeast Strains Providing For An Enhanced Rate Of The Fermentation Of Sugars, A Process To Obtain Such Yeasts And The Use Of These Yeasts. EP0306107 (A2).

OSINGA, KLAAS A; BEUDEKER, ROBERT F; VAN DER PLATT, JOHANNES B; D E HOLLANDER, JOHANNES A. (1993). Saccharomyces strains for maltose fermentation. U.S. Pat. No. 5,190,877 (A).

OYA KOZO; IWASAKI SUEO; HIRAKAWA KAN. (1988). Novel baker's yeast. JPS63294778 (A).

OYA KOZO; IWASAKI SUEO; HISADA YOJI; HIRAKAWA KAN. (1990). Baker's Yeast And Bread Dough Containing The Same Yeast. JPH0220284 (A).

PANADERO, J, RANDEZ-GIL, F, PRIETO, J A. (2005). Validation of a flour-free model dough system for throughput studies of baker's yeast. Applied And Environmental Microbiology 71(3): 1142-47.

ROGERS, DAVID, T; SZOSTAK, JACK, W. (1987). Yeast strains. WO8703006 (A1).

SAKURAI HIROAKI; OSAWA MASUMI; MATSUMOTO OSAMU. (2004). Method For Producing New Bread. JP2004229563 (A).

SALEMA-OOM M, DE SOUSA H, ASSUNCAO M, GONCALVES P, SPENCER-MARTINS I. (2011). “Derepression of a baker's yeast strain for maltose utilization is associated with severe deregulation of hxt gene expression”. J Appl Microbiol 110: 364-74.

SANO KOICHIRO; NIO NORIKI; MIWA TETSUYA; SEGURO KATSUYA. (2000). Production Of Freeze Resistant Yeast. JP2000037185 (A).

SANTE, L. (2017). Strain of the yeast species Saccharomyces cerevisiae, strains essentially derived from it and use thereof. US2017211159 (A1).

SIGLER, K. (2013). Acidification power test and similar methods for assessment and prediction of fermentation activity of industrial microorganisms. Kvasny Prum. (59):7-8.

STEENSELS, J, SNOEK, T, MEERSMAN, E, NICOLINO, M P, VOORDECKERS, K V E VERSTREPEN, K J. (2014). “Improving industrial Yeast Strains: Exploiting Natural And Artificial Diversity”. Fems Microbiol Rev 38: 947-95.

STRUYF, N, DER MAELEN, E V, HEMDANE, S, VERSPREET, J, VERSTREPEN, K J V E COURTIN, C M. (2017). “bread dough and baker's yeast: an uplifting synergy”. Comprehensive Reviews in Food Science And Food Safety 16: 850-67.

TAKADA ISATO; IZUMI TAKUYA; NODA NORIO. (2005). New Bread Yeast And Dough Containing The Same. JP2005245355 (A).

TAKADA ISATO; KYOGOKU KENJI; MATSUMOTO KEIJI; OYA KOZO; HARASHIMA TAKASHI. (1998). New Gene And New Yeast Containing The Same. JPH1066583 (A).

TAKADA ISATO; KYOGOKU KENJI; MATSUMOTO KEIJI; OYA KOZO; HARASHIMA TAKASHI. (1998). New gene and new yeast containing the same. JPH1066583 (A).

TAKAHASHI KIMIE; MOTOKI KENJI. (2005). Fused Yeast Producing Trehalose, Bread Dough Utilizing The Yeast And Method For Producing Bread. JP2005073622 (A).

TAKAHASHI KIMIE; NISHINA ATSURO; FUKUMOTO RYOHEI; MASUBUCHI TAKASHI. (2006). Fused Yeast. JP2006230298 (A).

TAKANO HIROYUKI; HINO AKIHIRO; ENDO HISANORI; NAKAGAWA NOBUAKI; SATO AKIO (1994). Frozen Bread Dough. JPH0670673 (A).

TAKANO HIROYUKI; HINO AKIHIRO; ENDO HISANORI; NAKAGAWA NOBUAKI; SATO AKIO. (1990). New Bread Yeast. JPH02238876 (A).

TAKANO HIROYUKI; NAKATOMI YASUO; NAKAJIMA RYOICHI; SUZUKI YASUO. (2000). New Excellent Bread Yeast. JP2000245438 (A).

TAMURA MASAHIKO; GOTO KUNIYASU; MORIYA HIROSHI; HASHIZUME KENICHI; TADENUMA MAKOTO; NISHITANI NAOMICHI. (1995). Low-Temperature Sensitive Baker's Yeast And Production Of Bread. JPH07213277 (A).

TAMURA MASAHIKO; MORIYA HIROSHI. (1997). Preparation Of Bread Yeast And Bread Baking Method. JPH09149785 (A).

TEUNISSEN A, DUMORTIER F, GORWA M -F, BAUER J, TANGHE A, LO″IEZ A, SMET P, VAN DIJCK P, THEVELEIN J M. (2002). “Isolation And Characterization Of A Freeze-Tolerant Diploid Derivative Of An Industrial Baker's Yeast Strain and Its Use in Frozen Doughs”. Appl Environ Microbiol 68: 4780-87.

VAN ROOIJEN, RUTGER JAN, BAANKREIS, RONALD, SCHOPPINK, PETER JOHANNES. (1999). Increased Production Of Carbon Dioxide By Yeast In Flour-Containing Dough. U.S. Pat. No. 5,968,790 (A).

WATANABE MAKOTO; FUKUDA KAZUO; ASANO KOZO. (1992). Preparation Of Food And Drink. JPH0491782 (A).

WICK, M, VANHOUTTE, J J, ADHEMARD, A, TURINI, G V E LEBEAULT J M. (2001). Automatic Method For Evaluating The Activity Of Sourdough Strains Based On Gas Pressure Measurements. Appl Microbiol Biotechnol 55:362-68.

XIAO DONGGUANG; WANG GUANGLU; DONG JIAN; ZHANG CUIYING. (2013). Freezing resistant Saccharomyces cerevisiae bacterial strain and construction method thereof. CN103232947 (A).

YAMAUCHI HIROAKI; TOMOFUJI IKUKO; KYOGOKU KENJI; MORITA KOJI; IWASAKI SUEO; OYA KOZO. (1995). Low-Temperature Sensitive Yeast And Bread Dough Using The Same Yeast. JPH07246087 (A).

ZHANG CUIYING; XIAO DONGGUANG; DONG WAN; SUN XI; WU MINGYUE. (2013). Quick-Fermentation Bread Yeast Strain And Breeding Method Thereof. CN103122323 (A).

ZHANG CUIYING; XIAO DONGGUANG; FENG BING; DONG JIAN; DU LIPING; CHEN YEFU (2014A). Saccharomyces cerevisiae Strain Suitable For Fermenting High-Sugar Dough And Construction Method Of Saccharomyces cerevisiae Strain. CN104212729 (A).

ZHANG CUIYING; XIAO DONGGUANG; FENG BING; DONG JIAN; DU LIPING; CHEN YEFU. (2014B). High-activity dry yeast applicable to high-sugar dough fermentation. CN104178435 (A).

ZHANG CUIYING; XIAO DONGGUANG; FENG BING; DONG JIAN; DU LIPING; CHEN YEFU. (2014C). Baker Yeast Strain Suitable For Fermentation Of Dough With High Sugar Content And Construction Method Thereof. CN104152363 (A).

ZHANG CUIYING; XIAO DONGGUANG; LIN XUE; DONG JIAN; BAI XIAOWEN; SONG HAIYAN. (2014D). High-Resistant Yeast Strain And Preparation Method Thereof. CN104017742 (A).

ZHENG D Q, CHEN J, ZHANG K, GAO K -H, LI O, WANG P M, ZHANG X Y, DU F G, SUN P Y & QU A M. (2013). “Genomic structural variations contribute to trait improvement during whole-genome shuffling of yeast. Appl Microbiol Biotechnology 98: 1-12.

1. A method of development and selection of the baker's yeast strains that can exhibit high raising activity in sweet and/or lean dough in the bakery and pastry industries, characterized by comprising the following method steps;
i. developing the baker's yeast strain, preferably with the process of multiple crossing, by using the combination of one or more than one of the mutagenesis, evolutionary engineering, asexual hybridization, recombinant DNA technology and sexual hybridization methods;
ii. preparing the synthetic or flour-based selective nutrient medium, preferably the synthetic nutrient medium with the sugar content of 0%, 15% and/or 25% and incubating the strains developed, in multiple well plates inside the nutrient medium prepared;
iii. scanning the strains incubated by using a high-resolution optical scanner, or by naked eye or a device with a spectrophotometric reader, preferably by using a high-resolution optical scanner, and selecting the strains that perform the best acidification which is to be determined based on the results of the analysis.

2. The method according to claim 1, wherein the synthetic selective nutrient medium with the sugar content of 0% which is mentioned in the method step of (ii) comprises 7.00% (w/v) maltose indicator nutrient medium, 13.00% (w/v) sorbitol, 0.24% (w/v) calcium-propionate, 0.90% (v/v) pH indicator, 78.86% (v/v) water.

3. The method according to claim 2, wherein said maltose indicator nutrient medium is obtained by mixing the components of 1% (w/v) yeast extract, 2% (w/v) peptone; 2% (w/v) maltose and 2% agar (w/v) in distilled water.

4. The method according to claim 1, wherein the synthetic selective nutrient medium with the sugar content of 15% which is mentioned in the method step of (ii) comprises 20% (v/v) concentrated nutrient solution, 0.50% (w/v) yeast extract, 3.00% (w/v) glucose, 9.00% (w/v) maltose, 10.00% (w/v) sorbitol, 15.00% (w/v) sucrose, 2.00% (w/v) agar, 0.24% (w/v) calcium-propionate, 0.90% (v/v), pH indicator, 39.36% (v/v) water.

5. The method according to claim 1, whrerein the synthetic selective nutrient medium with the sugar content of 25% which is mentioned in the method step of (ii) comprises 20% (v/v) concentrated nutrient solution, 0.50% (w/v) yeast extract, 3.00% (w/v) glucose, 9.00% (w/v) maltose, 16.00% (w/v) sorbitol, 25.00% (w/v) sucrose, 2.00% (w/v) agar, 0.24% (w/v) calcium-propionate, 0.90% (v/v), pH indicator, 23.36% (v/v) water.

6. The method according to claim 4, wherein said concentrated nutrient solution is obtained by dissolving the components of 2% (w/v) MgSO4.7H2O (magnesium sulfate hepta-hydrate), 0.8% (w/v) KCl (potassium chloride), 4.7% (w/v) (NH4)2HPO4 (ammonium phosphate dibasic), 0.0016% (w/v) Thiamine, 0.0016% (w/v) Pyridoxine, 0.016% (w/v) Nicotinic acid in a 0.75M Citrate Buffer; by setting the final pH of the solution to 5.5 with the addition of NaOH (sodium hydroxide); by being sterilized through a 0.2 μm sterilized filter.

7. The method according to claim 1, wherein the flour-based selective nutrient medium having a sugar content of 0% which is mentioned in the method step of (ii) comprises 60.52% (w/w) wheat flour, 0.91% (w/w) salt, 0.24% (w/w) calcium-propionate, 38.33% (v/w) water.

8. The method according to claim 1, whrerein the flour-based selective nutrient medium having a sugar content of 15% which is mentioned in the method step of (ii) comprises 58.74% (w/w) wheat flour, 8.81% (w/w) sugar, 3.92% (w/w) margarine, 0.88% (w/w) salt, 0.23% (w/w) calcium-propionate, 27.41% (v/w) water.

9. The method according to claim 1, whrerein the flour-based selective nutrient medium having a sugar content of 25% which is mentioned in the method step of (ii) comprises 56.53% (w/w) wheat flour, 14.13% (w/w) sugar, 5.65% (w/w) margarine, 0.85% (w/w) salt, 0.23% (w/w) calcium-propionate, 22.61% (v/w) water.

10. The method according to claim 1, wherein the multiple well plates which are mentioned in the method step of (ii) are 1, 4, 6, 8, 12, 16, 24, 48, 96, 384 and 1536-well plates, preferably 48-well plates.

11. The method according to claim 1, whrerein the strains which are incubated in the method step of (ii) are kept in incubation for 10 minutes to 4 days at the temperature of 0° C. and +35° C.; preferably for 2-3 days at +30° C.

12. The method according to claim 5, wherein said concentrated nutrient solution is obtained by dissolving the components of 2% (w/v) MgSO4.7H2O (magnesium sulfate hepta-hydrate), 0.8% (w/v) KCl (potassium chloride), 4.7% (w/v) (NH4)2HPO4 (ammonium phosphate dibasic), 0.0016% (w/v) Thiamine, 0.0016% (w/v) Pyridoxine, 0.016% (w/v) Nicotinic acid in a 0.75M Citrate Buffer; by setting the final pH of the solution to 5.5 with the addition of NaOH (sodium hydroxide); by being sterilized through a 0.2 μm sterilized filter.
